(19) United States
(12) Patent Application Publication
(10) Pub. No.: US 2024/0415742 A1
(43) Pub. Date: Dec. 19, 2024

(54) Bioactive Dental Restorative Material

(71) Applicant: MUSSEL POLYMERS, INC., Bethlehem, PA (US)

(72) Inventor: Eric Anderson, Princeton, NJ (US)

(73) Assignee: Mussel Polymers, Inc., Bethlehem, PA (US)

(21) Appl. No.: 18/718,982

(22) PCT Filed: Dec. 13, 2022
(86) PCT No.: PCT/US2022/081438

§ 371 Date: Jun. 12, 2024

Related U.S. Application Data
(60) Provisional application No. 63/288,828, filed on Dec. 13, 2021.

(51) Int. Cl. A61K 6/836; A61K 6/15; A61K 6/20; A61K 6/887; A61K 6/889

(52) U.S. Cl. CPC A61K 6/836; A61K 6/15; A61K 6/20; A61K 6/887; A61K 6/889

(57) ABSTRACT

Conventional dental cements lack certain properties such as the facilitation of optimal re-mineral-ization. The novel bioactive glass-ionomer dental and bone cement formulations disclosed herein induces mineralization at the in vivo interface and exhibits superior handling properties (shorter setting time) and mechanical properties (improved bond strength to dentin and flexural strength). The present invention presents the first bioactive glass-ionomer dental cement with mineralization power, exhibiting improved bond strength to tooth structure, sharp setting time and superior me-chanical strength.

### CROSS REFERENCE TO RELATED APPLICATIONS

This application claims the benefit of U.S. Provisional Application No. 63/288,828 filed Dec. 13, 2021, the entirety of which are incorporated by reference herein.

### FIELD OF THE INVENTION

The present invention relates to dental adhesive compositions and methods for making and using them.

### BACKGROUND

Glass-ionomer cements (GICs) are known to be useful as dental restorative materials. GICs are formed from glass ionomer powders which comprise finely ground ceramic powders, the main components of which are silica (SiO₂), alumina (Al₂O₃) and calcium fluoride (CaF₂) as flux, sodium fluoride (NaF) and cryolite (Na₃AlF₆) or aluminum phosphate (AlPO₄). Phosphate and fluoride salts are used to modify and control the setting characteristics of the cement. GICs are, in general, formed predominantly from alumina and silica, which form the back-bone of the glass. Glass-ionomer cements are formed by the reaction of an ion-leachable alumino-silicate glass powder with an aqueous solution of polyacid such as polyacrylic acid (polyalkanoic acid). GICs are well-known for their properties of direct adhesion to tooth structure and base metals; for anti carcinogenicity due to release of fluoride; low shrinkage resulting in minimized microleakage at the tooth-enamel interface; biological compatibility and low cytotoxicity.

However, conventional GICs suffer from a number of disadvantages such as lack of re-mineralization properties, low bond strength to tooth structure, long setting time, brittleness, poor compressive strength and poor fracture resistance, all of which limit their utility. Consequently, there is a need to improve the biological and physical properties of glass ionomer cements (GICs).

### SUMMARY OF THE INVENTION

In certain aspects, the present invention provides a glass ionomer cement composition comprising fluoroaluminosilicate glass particles coated with a catechol-containing thin-film comprising monomeric, oligomeric, and/or polymeric catechol, semi-quinone, and/or quinone.

In some embodiments, the catechol-containing thin film comprises poly catechol-styrene. In some embodiments, the thickness of the catechol-containing thin-film ranges between 5 and 100 nanometers in thickness. In some embodiments, the glass further comprises calcium, sodium, phosphate or a combination thereof. In some embodiments, the catechol-containing thin-film comprises poly-catechol-styrene HCl.

In some embodiments, the glass ionomer cement composition further comprises fluoroaluminosilicate glass particles not coated by a catechol-containing thin-film. In some embodiments, the relative amount of fluoroaluminosilicate glass particles coated by a catechol-containing thin-film is from 1% to 95% of the total fluoroaluminosilicate glass particles present in the composition. In some embodiments, the relative amount of fluoroaluminosilicate glass particles coated by a catechol-containing thin-film is from 1% to 50% of the total fluoroaluminosilicate glass particles present in the composition. In some embodiments, the relative amount of fluoroaluminosilicate glass particles coated by a catechol-containing thin-film is from 8% to 30% of the total fluoroaluminosilicate glass particles present in the composition.

In some embodiments, the glass ionomer cement composition further comprises polyacid. In some embodiments, said polyacid is selected from polyacrylic acid, itaconic acid, maleic acid, tartaric acid or any combination thereof.

In some embodiments, glass ionomer cement composition further comprises a resin. In some embodiments, the resin is selected from HEMA (hydroxyethyl methacrylate), Bis-GMA (bisphenol A-glycidyl methacrylate), TEGMA (triethylene glycol dimethacrylate) or UDMA (urethane di-methacrylate resin) or any combination thereof. In some embodiments, the catechol-containing film-coated or the poly-catechol-styrene-coated fluoroaluminosilicate glass particles are disposed within said hydroxyethyl methacrylate resin. In some embodiments, the hydroxyethyl methacrylate resin is light cured.

In some embodiments, the glass ionomer cement composition exhibits a setting time of less than 5 minutes at 25° C.

In certain aspects, the present invention provides a method of using the glass ionomer cement composition as contemplated herein in dentistry, the method comprising: a) mixing said fluoroaluminosilicate glass particles coated by a catechol-containing thin-film with said fluoroaluminosilicate glass particles not coated by a catechol-containing thin-film to form a powder mixture; b) disposing the formed mixture at an in vivo site.

In some embodiments, the method further comprises mixing the powder comprising a catechol-containing thin-film-coated glass particles with a liquid comprising polyacid to form a cement; loading a crown or restoration with said cement; and placing said crown or restoration in a patient's mouth; such that said cement composition acts as a luting element.

In some embodiments, the method further comprises applying said cement to a tooth such that said cement com-position acts as a material to bond to carious lesions, a material to reduce teeth sensitivity, as a material to promote mineralization of teeth white spots or to any combination thereof.

In certain aspects, the present invention provides a method of using the glass ionomer cement composition comprising calcium, sodium, phosphate, or a combination thereof to generate a layer of hydroxyapatite at the surface of a tooth. In some embodiments, said method comprises disposing the catechol-containing thin-film-coated fluoroaluminosilicate glass particles in a resin to form a resin/particle composition; applying the resin/particle composition to said surface of said tooth.

In some embodiments, the methods further comprise curing the resin/particle composition by light irradiation.

In certain aspects, the present invention provides a glass ionomer cement composition comprising fluoroaluminosilicate glass particles coated with a polymeric thin-film comprising monomeric, oligomeric, and/or polymeric catechol, semi-quinone, and/or quinone.

In some embodiments, the polymeric thin film comprises poly catechol-styrene. In some embodiments, the thickness of the polymeric thin-film ranges between 5 and 100 nanometers in thickness.

In some embodiments, the glass ionomer cement composition comprising fluoroaluminosilicate glass particles coated with a polymeric thin-film further comprises fluoroaluminosilicate glass particles not coated by a polymeric thin-film. In some embodiments, the relative amount of fluoroaluminosilicate glass particles coated by a polymeric thin-film is from 1% to 95% of the total fluoroaluminosilicate glass particles present in the composition.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows an illustration of the fabrication of poly-catechol-styrene (PCS) coated glass particles and PCS containing glass ionomer cements. It will be appreciated that for simplicity and clarity of illustration, elements shown in the figures have not necessarily been drawn to scale. For example, the dimensions of some of the elements may be exaggerated relative to other elements for clarity.

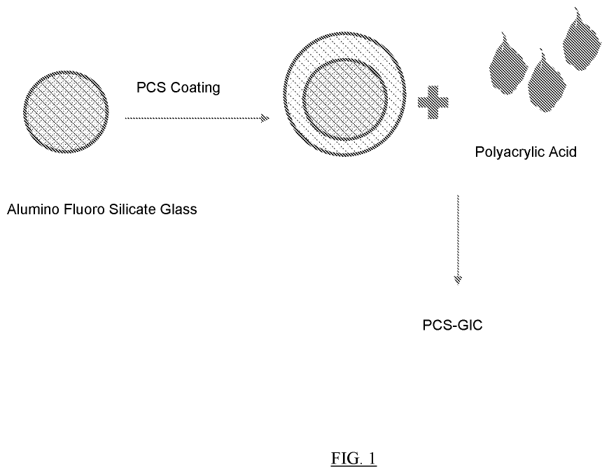

### DETAILED DESCRIPTION OF THE PRESENT INVENTION

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

In the present disclosure, the term “subject” includes any human or non-human animal. In certain embodiments, the subject is a human or non-human mammal. In certain embodiments, the subject is a human.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the disclosure which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the disclosure that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

Glass ionomer cements (GIC) were invented by Wilson et al. at the Laboratory of the Government Chemist in 1969. These materials are water-based cements also known as polyalkenoate cements. They are based on the reaction between an alumino-silicate glass and polyacrylic acid, and cement formation arises from the acid-base reaction between the components. The glass ionomer name is derived from the formulation of the glass powder and the ionomer that contains carboxylic acids. These cements are adhesive to tooth structure and are translucent. The matrix of the set cement is an inorganic-organic network with a highly cross-linked structure. The first glass ionomer cement (GIC) introduced had the acronym “ASPA” and comprised alumina-silicate glass as the powder and polyacrylic acid as the liquid. This product was first sold in Europe (De Trey Company and Amalgamated Dental Company) and later in the USA. Glass ionomer cements have desirable properties, such as adhesion to moist tooth structure and an anti-cariogenic action (due to fluoride release). In addition, the coefficient of thermal expansion for glass ionomers is close to that of tooth structure and they are biocompatible. Because of these unique properties, GICs are very useful and important as dental restorative materials.

However, in addition to their advantages, GICs suffer from a number of disadvantages such as lack of restoration properties, low bond strength to tooth structure, long setting time, brittleness, poor compressive strength and poor fracture resistance, all of which limit their utility. Therefore, there is a need to improve the biological and physical properties of glass-ionomer cements (GICs).

In the present invention, a novel bioactive dental restorative material is introduced. This new dental material is based on glass-ionomer cements with restoration capability. Moreover, this new glass-ionomer cement formulation exhibits enhanced physical (fast setting time) and mechanical strength and enhanced adhesion to tooth structure for applications in dentistry and orthopedics.

In an aspect, the disclosure is directed to a polymeric material comprising a catechol containing polymer or oligomer, wherein said catechol presents as a catechol and/or as a semi-quinone and/or as a quinone without the presence of a primary amine or a secondary amine; and wherein the polymeric material optionally comprises at least one of: a) a reactive species separate from the catechol containing polymer or oligomer; and b) a catalyst, co-catalyst or an accelerator.

In some embodiments, the polymeric material comprises the catechol containing polymer or oligomer, wherein said catechol presents as a catechol and/or as a semi-quinone and/or as a quinone without the presence of a primary amine or a secondary amine and wherein the polymeric layer also comprises a reactive material that is not reactive with catechol or quinone.

In some embodiments, the polymeric material comprises the catechol containing polymer or oligomer, wherein said catechol presents as a catechol and/or as a semi-quinone and/or as a quinone without the presence of a primary amine or a secondary amine; and also comprises the reactive species separate from the catechol containing polymer or oligomer.

In some embodiments, the polymeric material comprises the catechol containing polymer or oligomer, wherein said catechol presents as a catechol and/or as a semi-quinone and/or as a quinone without the presence of a primary amine or a secondary amine. In some embodiments, the polymeric material comprises the catechol containing polymer or oligomer, wherein said catechol presents as a catechol and/or as a semi-quinone and/or as a quinone without the presence of a primary amine or a secondary amine; and also comprises the catalyst, co-catalyst or an accelerator.

In some embodiments, the polymeric material comprises the catechol containing polymer or oligomer, wherein said catechol presents as a catechol and/or as a semi-quinone and/or as a quinone without the presence of a primary amine or a secondary amine. In some embodiments, the polymeric material comprises the catechol containing polymer or oligomer, wherein said cat-echol presents as a catechol and/or as a semi-quinone and/or as a quinone without the presence of a primary amine or a secondary amine; and also comprises both the reactive species separate from the catechol containing polymer or oligomer and the catalyst, co-catalyst or an accelerator.

In an aspect, the disclosure is directed to a polymeric layer comprising a catechol containing monomer, polymer, or oligomer, wherein said catechol presents as a catechol and/or as a semi-quinone and/or as a quinone without the presence of a primary amine or a secondary amine; and wherein the polymeric layer also comprises a reactive material that is not reactive with catechol or quinone; and wherein the polymeric layer further comprises a bulk adhesive layer disposed adjacent to and in contact with the polymeric layer.

In some embodiments, the catechol containing monomer, polymer, or oligomer in the polymeric layer is monomeric. In some embodiments, the catechol containing monomer, polymer, or oligomer in the polymeric layer is oligomeric. In some embodiments, the catechol containing monomer, polymer, or oligomer in the polymeric layer is polymeric.

In some embodiments, the polymeric layer comprises the reactive species separate from the catechol or catechol containing material; and the reactive species is an acrylic such as 2,2-bis[4-(2-hydroxy-3-methacryloyloxypropoxy)phenyl]propane (BisGMA), ethoxylated bisphenol-A dimethacrylate (EBPADMA), triethylene glycol dimethacrylate (TEGDMA), urethane di-methacrylate (UDMA), tert-butylphenoxy BisGMA (MtBDMA), modified urethane dimethacrylate, amide modified bisphenol-A, CH3BisGMA, acidic bisphenol-A dimethacrylate, dimethacrylate from cycloaliphatic epoxide, aromatic urethane dimethacrylate, urethane modified BisGMA, acid aromatic dimethacrylate, oxydiphthalic-acid dimethacrylate, phenyl dihydroxymethacrylate diphosphonate, Acidic Bisphenol-A dimethacrylate, morpholine carbonyl methacrylate, phenyl carbonate methacrylate.

In some embodiments, the polymeric layer includes free radical polymerization initiators such as acrylate polymerization initiators, including those that are light activated, such as benzoyl peroxide (BPO), 2,3-bornanedione (Camphorquinone), Ethyl-4-(dimethylamino)benzoate (ED-MAB), 2-(Ethylhexyl)-4-(dimethylamino)benzoate (ODMAB), 2-(Ethylhexyl)-4-(dimethylamino)benzoate (TPO), Diphenyl(2,4,6-trimethylbenzoyl)-phosphineoxide or combinations thereof.

In some embodiments, the acrylate is an acrylate monomer comprising a vinyl group and at least one of a carboxylic acid ester and a carboxylic acid nitrile; and wherein the acrylate is linear or branched. In some embodiments, the acrylate is ethyl acrylate, ethylene-methyl acrylate, methyl methacrylate, 2-chloroethyl vinyl ether, 2-hydroxyethyl acrylate, hydroxyethyl methacrylate, butyl acrylate, trimethylolpropane triacrylate (TMPTA) or combinations thereof.

In an aspect, the polymeric layer has a thickness of from about 10 nanometers to about 100 microns. In some embodiments, the polymeric layer has a thickness of from about 15 nanometers to about 50 microns. In some embodiments, the polymeric layer has a thickness of from about 15 nanometers to about 15 microns. In some embodiments, the polymeric layer has a thickness of from about 150 nanometers to less than about 15 microns. In some embodiments, the polymeric layer has a thickness of from about 150 nanometers to about 1.5 microns.

In some embodiments, the polymeric layer has a thickness of from about 10 nanometers to about 100 microns; or from about 10 nanometers to about 100 nanometers; or from about 100 nanometers to about 150 nanometers; or from about 150 nanometers to about 200 nanometers; or from about 200 nanometers to about 250 nanometers; or from about 250 nanometers to about 300 nanometers; or from about 300 nanometers to about 350 nanometers; or from about 350 nanometers to about 400 nanometers; or from about 400 nanometers to about 450 nanometers; or from about 450 nanometers to about 500 nanometers; or from about 500 nanometers to about 550 nanometers; or from about 550 nanometers to about 600 nanometers; or from about 600 nanometers to about 650 nanometers; or from about 650 nanometers to about 700 nanometers; or from about 700 nanometers to about 750 nanometers; or from about 750 nanometers to about 800 nanometers; or from about 800 nanometers to about 850 nanometers; or from about 850 nanometers to about 900 nanometers; or from about 900 nanometers to about 950 nanometers; or from about 950 nanometers to about 1000 nanometers.

In some embodiments, the polymeric layer has a thickness of from about 1 micron to about 1.5 microns; or from about 1.5 microns to about 5 microns; or from about 5 microns to about 10 microns; or from about 10 microns to about 15 microns; or from about 15 microns to about 20 microns; or from about 20 microns to about 25 microns; or from about 25 microns to about 30 microns; or from about 30 microns to about 35 microns; or from about 35 microns to about 40 microns; or from about 40 microns to about 45 microns; or from about 45 microns to about 50 microns; or from about 50 microns to about 55 microns; or from about 55 microns to about 60 microns; or from about 60 microns to about 65 microns; or from about 65 microns to about 70 microns; or from about 70 microns to about 75 microns; or from about 75 microns to about 80 microns; or from about 80 microns to about 85 microns; or from about 85 microns to about 90 microns; or from about 90 microns to about 95 microns; or from about 95 microns to about 100 microns.

In an aspect, the catechol containing monomer, polymer or oligomer in the polymeric material comprises poly-catechol styrene (PCS).

In some embodiments, the PCS comprises a solution containing from about 0.001% to 10% PCS, from about 0.05% to about 5% PCS, from about 0.01% to about 2% PCS, from about 0.5% to about 1% PCS, from about 0.1% to about 0.5% PCS and any and all increments therebetween. In some embodiments, the PCS comprises about 0.1% catechol.

In some embodiments, the PCS comprises from about 20% catechol to about 22% catechol; or from about 22% catechol to about 24% catechol; or from about 24% catechol to about 26% catechol; or from about 26% catechol to about 28% catechol; or from about 28% catechol to about 30% catechol; or from about 30% catechol to about 32% catechol; or from about 32% catechol to about 34% catechol; or from about 34% catechol to about 36% catechol; or from about 36% catechol to about 38% catechol; or from about 38% catechol to about 40% catechol.

In some embodiments, the PCS comprises from about 15% catechol to about 50% catechol. In some embodiments, the PCS comprises from about 20% catechol to about 40% catechol. In some embodiments, the PCS comprises from about 25% catechol to about 35% catechol. In some embodiments, the PCS comprises about 25% catechol. In some embodiments, the PCS comprises about 35% catechol.

In some embodiments, the PCS comprises from about 20% catechol to about 22% catechol; or from about 22% catechol to about 24% catechol; or from about 24% catechol to about 26% catechol; or from about 26% catechol to about 28% catechol; or from about 28% catechol to about 30% catechol; or from about 30% catechol to about 32% catechol; or from about 32% catechol to about 34% catechol; or from about 34% catechol to about 36% catechol; or from about 36% cate-chol to about 38% catechol; or from about 38% catechol to about 40% catechol.

In some embodiments, the PCS is prepared in one or more suitable solvents. For example, the PCS may be prepared as a solution in acetone, tert-butyl alcohol, ethanol, isopropyl alcohol, or a combination thereof, or one or more other suitable solvents as understood in the art. In some embodiments, the PCS is prepared as a solution in acetone. In some embodiments, the PCS is prepared as a solution in tert-butyl alcohol. In some embodiments, the PCS is prepared as a solution in isopropyl alcohol. In some embodiments the PCS is prepared as a solution in ethanol.

In some embodiments, the polymeric layer comprises a reactive material that is not reactive with catechol or quinone. In some embodiments, the reactive material is not reactive at ambient temperature with catechol or quinone. In some embodiments, the reactive material is not reactive at low temperature with catechol or quinone.

In some embodiments, the reactive material that is not reactive with catechol or quinone is a resin, an oligomer, a polymer, or a monomer. In some embodiments, the reactive material an oligomer. In some embodiments, the reactive material a polymer. In some embodiments, the reactive material a monomer.

In an aspect, the polymeric layer is a continuous layer. In an aspect, the polymeric layer is a non-continuous layer. In an aspect, the polymeric layer is a patterned layer or a textured layer.

The bioactive dental restorative material with restorative properties is discussed in terms of poly catechol-styrene. However, the other materials formed from catechol containing polymeric material can also be used for the same purposes.

The present invention relates to synthesis of novel poly-catechol-styrene (PCS)-modified fluoroaluminosilicate glass bio-ceramic particles and their applications in dentistry and orthopedics. This novel glass-ionomer formulation stimulates mineral hydroxyapatite formation and the natural re-mineralization process at the cement tooth interface, induces tertiary dentin formation, and reduces sensitivity. Additionally, due to the presence of PCS, the developed cement exhibit superior bond strength to dentin tooth structure, presents with a sharp setting time and superior mechanical properties. This novel glass-ionomer dental cement can be used as a restorative material, cavity liner, or a luting cement. Additionally, since it can regenerate a layer of hydroxyapatite on the surface of the tooth and bonds to tooth structure it can be used for direct/indirect pulp capping procedures.

In the following detailed description, numerous specific details are set forth in order to provide a thorough understanding of the invention. However, it will be understood by those skilled in the art that the present invention may be practiced without these specific details. In other in-stances, well-known methods, procedures, and components have not been described in detail so as not to obscure the present invention.

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

In one embodiment, this invention provides a glass ionomer cement composition comprising fluoroaluminosilicate glass particles coated by poly-catechol-styrene (PCS). In one embodiment, the thickness of the poly-catechol-styrene coating layer ranges between 5 and 100 nanometers in thickness.

In one embodiment, the composition further comprising a fluoroaluminosilicate glass particles not coated by poly-catechol-styrene. In one embodiment, the relative amount of fluoroaluminosilicate glass microparticles coated by poly-catechol-styrene is from 1% to 100% of the total fluoroaluminosilicate glass microparticles present in the composition. In one embodiment, the relative amount of fluoroaluminosilicate glass microparticles coated by poly-catechol-styrene in the composition is from 1% to 50%, from 1% to 95% or from 8% to 30% of the total amount of fluoroaluminosilicate glass microparticles in the composition.

In one embodiment, the composition further comprising polyacid. In one embodiment, the polyacid is selected from polyacrylic acid, itaconic acid, maleic acid, tartaric acid or any combination thereof.

In one embodiment, the composition further comprising a resin. In one embodiment, the resin is selected from HEMA (hydroxyethyl methacrylate), Bis-GMA (bisphenol A-glycidyl methacrylate), TEGMA (triethylene glycol dimethacrylate) or UDMA (urethane di-methacrylate resin) or any combination thereof. In one embodiment, the PCS-coated fluoroaluminosilicate glass particles are disposed within the hydroxyethyl methacrylate resin (or in any other resin). In one embodiment, the hydroxyethyl methacrylate resin (or any other resin) is light cured.

In one embodiment, the composition exhibits a setting time of less than 5 minutes at 25° C. In one embodiment, the poly-catechol-styrene comprises poly-catechol-styrene HCl. In one embodiment, the glass further comprises calcium, sodium or a combination thereof.

In one embodiment, this invention provides a method of using the cement composition as described herein above in dentistry, the method comprising: mixing the fluoroaluminosilicate glass particles coated by poly-catechol-styrene (PCS) with the fluoroaluminosilicate glass particles not coated by poly-catechol-styrene (PCS) to form a powder mixture; disposing the mixture at an in vivo site.

In one embodiment, rinsing the cavity is performed with water, i.e., the cavity is rinsed with water. In one embodiment, after rinsing with water, and prior to placing the PCS-containing GIC directly in the prepared cavity, the cavity is dried.

In one embodiment, rinsing the cavity is followed by drying. In one embodiment, the cavity is rinsed with water and then dried.

In one embodiment, the method of using the cement composition in dentistry further comprises: mixing the powder comprising PCS-coated glass particles with a liquid comprising polyacid to form a cement; load a crown or restoration with the cement; place the crown or restoration in a patient's mouth; such that the cement composition acts as a luting element.

In one embodiment, the method of using the cement composition in dentistry further comprising applying the cement to a tooth such that the cement composition acts as a material to bond to carious lesions, a material to reduce teeth sensitivity, as a material to promote mineralization of teeth white spots or to any combination thereof.

In one embodiment, this invention provides a method of using the particle composition as described herein above to generate a layer of hydroxyapatite at the surface of a tooth, the method comprising: disposing the PCS-coated fluoroaluminosilicate glass particles in a resin; applying the composition to white spots on the surface of the tooth. curing the composition by light irradiation.

According to this aspect and in one embodiment, the step of disposing the particles in the resin comprises disposing a powder of the particles comprising PCS-coated GIC particles in the resin. The particles are mixed with the resin in one embodiment.

In one embodiment, the method further comprising extraction of calcium and phosphate from a patient saliva by said composition and initialization of restoration.

In one embodiment, the composition application step is being conducted by a physician or by a patient. In one embodiment, the composition in an unfilled resin is supplied as an over-the-counter (OTC) product. In one embodiment, the OTC product comprises a pre-loaded tray with PCS-coated glass-ionomer-cement particles in an unfilled resin.

In some embodiments, the step of curing by light is optional. According to this aspect and in one embodiment no light-curing is performed. For example, an OTC product does not require light curing in one embodiment, while the office version of the product is light curable in one embodiment. In other embodiments, the office preparation does not require light curing.

In embodiments of this invention, GIC compositions of the invention comprise glass particles (coated by PCS, or uncoated, or a combination thereof), acidic polymer or acidic copolymer or a combination thereof. In some embodiments, compositions of this invention comprise a resin. The weight ratio of glass particles to acidic polymer and/or to the resin when incorporated can be any ratio within a range known in the art. In one embodiment, the polyacid(s) used to form the GIC are selected from polyacrylic acid, polylactic acid, polyitaconic acid, any polyalkenoic acid, tartaric acid or any combination thereof.

In one embodiment, the powder is or comprises fluoro-alumina-silicate particles coated with PCS. In one embodiment, the liquid is or comprises a copolymer of acrylic acid and itaconic acid.

The invention disclosed herein has a number of embodiments. A typical embodiment of the invention is a glass ionomer cement composition comprising fluoroaluminosilicate glass particles coated by catechol-styrene (e.g., catechol-styrene hydrochloride), optionally in combination with fluoroaluminosilicate glass microparticles not coated by catechol-styrene hydrochloride. In such compositions, the relative amounts of fluoroaluminosilicate glass microparticles coated by catechol-styrene hydrochloride can be from 1% to essentially 100% of the total fluoroaluminosilicate glass microparticles present in the composition (e.g., where 1% to 50%, 1% to 95%, 1% to 100%, 5% to 95%, 5% to 100%, 8% to 30%, 30% to 80%, etc., of the fluoroaluminosilicate glass microparticles in the composition are coated by catechol-styrene hydrochloride while the other fluoroaluminosilicate glass microparticles in the composition are not coated by poly-catechol-styrene) In one embodiment, the poly-catechol-styrene is coated on the fluoroaluminosilicate glass microparticles to form a layer that is between 5 and 50 nanometers in thickness. These fluoroaluminosilicate glass microparticles can form a powder component of a multi-component mixture, for example a mixture that includes this first powder component in combination with a second liquid component, for example one that includes a polyacid such as polylactic acid.

In these multicomponent glass ionomer cement compositions, the PCS coating the fluoroaluminosilicate glass microparticles is included in amounts and disposed on the microparticles in a way that makes the composition having a number of desirable physical/material qualities. For example, in some embodiments of the invention, the composition comprising the mixture of fluoroaluminosilicate glass microparticles including particles coupled to PCS exhibits a compressive strength that is at least 10% greater (e.g., 10%-35% greater) than the compressive strength observed with a control/comparative composition that does not comprise fluoroaluminosilicate glass microparticles coupled to PCS hydrochloride. In some embodiments of the invention, the composition comprising the mixture of fluoroaluminosilicate glass microparticles including particles coupled to PCS exhibits a flexural strength that is at least 10% greater (e.g., at least 2× greater) than the flexural strength observed with a control/comparative composition that does not comprise fluoroaluminosilicate glass microparticles coupled to PCS hydrochloride. In some embodiments of the invention, the composition comprising the mixture of fluoroaluminosilicate glass microparticles including particles coupled to PCS exhibits a shear bond strength that is at least 10% greater (e.g., at least 2 times. greater) than the shear bond strength observed with a control/comparative composition that does not comprise fluoroaluminosilicate glass microparticles coupled to PCS hydrochloride. In some embodiments of the invention, the composition comprising the mixture of fluoroaluminosilicate glass microparticles including particles coupled to PCS exhibits a hardness after 7 days that is at least 10% greater than the hardness observed with a control/comparative composition that does not comprise fluoroaluminosilicate glass microparticles coupled to PCS hydrochloride. In some embodiments of the invention, the composition comprising the mixture of fluoroaluminosilicate glass microparticles including particles coupled to PCS exhibits an adhesion to dentin (bonding to substrate) that is at least 2× the adhesion observed with a control/comparative composition that does not comprise PCS hydrochloride.

In some embodiments of the invention the compositions of the invention facilitate hydroxyapatite restoration, for example so that after 7 days following application of the composition to a surface of a tooth, hydroxyapatite restoration is observed. Typically, this hydroxyapatite restoration is at least 10% (e.g., at least 100%) greater than hydroxyapatite restoration observed with a control/comparative composition that does not comprise PCS hydrochloride. In some embodiments of the invention, the compositions of the invention are disposed within a hydroxyethyl methacrylate resin that is curable with light. In certain embodiments of the invention, the composition exhibits a setting time of less than 5 minutes at 25° C.

In certain embodiments of the invention, the compositions of the invention are used to generate a layer of hydroxyapatite at the surface of a tooth, wherein after 7 days, hydroxyapatite restoration is at least 10% (e.g., at least 100%) greater than hydroxyapatite restoration observed with a control/comparative composition that does not comprise PCS hydrochloride.

In one embodiment, this invention relates to dental adhesive hydrogel compositions and methods for making and using them.

Without being bound to any theory, it is believed that the hydroxyl and the amine groups in the PCS coating interact with the acid, leading to increased acid-base reaction and increase in the mechanical properties of the GIC in some embodiments.

A major advantage of using a resin in compositions of this invention is that it makes it visible light crosslinkable. Any photocrosslinkable resin can be used in embodiments of this invention, such as bis-GMA (bisphenol A-glycidyl methacrylate), TEGMA (triethylene glycol di-methacrylate), UDMA (urethane di-methacylate resin), or HEMA (hydroxyethyl methacrylate).

In one embodiment, when the particle composition is incorporated in a resin, light is applied to the composition/resin after incorporation, for resin cross linking. The resin comprising the composition can be exposed to a ‘blue light’ lamp. The resin comprising the composition can be cured by any electromagnetic irradiation source. The curing wavelength of the irradiation source is chosen to fit the specific resin employed. The curing wavelengths for each resin/polymer are known in the art. The curing wavelength can be in the UV range, in the visible range or in other spectral ranges as required by a certain resin. Crosslinkable means that the resin is capable of forming cross-linkages (capable of becoming cross-linked) upon exposure to light/irradiation.

In one embodiment, acid-base reaction triggers setting of the cement. Acid is provided by the polyacid in the liquid and the base is the glass in the glass particles in one embodiment.

In embodiments of this invention, the cement composition acts as a restorative material, a luting cement, a pulp capping, a material to reduce teeth sensitivity, as a material to promote mineralization of teeth or as any combination thereof.

In some embodiments, this invention provides a method of using GIC compositions of this invention to generate a layer of hydroxyapatite at the surface of a tooth, the method comprising applying the composition to a surface of a tooth restoration.

Particles coated by PCS are sometimes refer to as particles coupled to PCS. In embodiments of this invention, particles are fully coated by PCS. In other embodiments, particles are partially coated by PCS. In embodiments, clusters or aggregates of particles are coated (fully or partially) by PCS. Embodiments of this of this invention includes collections of particles coated by PCS wherein the particles are fully coated, partially coated or wherein some particles are fully coated while others are partially coated. All such combinations in some embodiments, are mixed with particles that are not coated by PCS, to form compositions of particles of this invention. In other embodiments, the PCS coated particles (fully or partially or combinations thereof) are used in compositions of this invention without additional non-PCS-coated particles. According to this aspect and in one embodiment, the collection of coated particles (partially or fully or partially and fully) is not mixed with a collection of non-coated particles before mixing with the liquid acid.

In some embodiments, the glass particles used are FAS, or comprise FAS. FAS is fluoroaluminosilicate. Other glasses may be used, e.g., glasses that do not comprise fluoride and/or aluminum ions or glasses that comprise any ion/atom combination of calcium, sodium, phosphorous, fluoride, aluminum, iron, silicon. Embodiments described herein for FAS are applicable to any other glass from the glasses described herein above.

GIC refers to glass ionomer cement. The term ‘ionomer’ relates to the polyacid used to form the cement. However, the glass particles are also referred to as ‘GIC particles’ as known in the art and in view of their use in the formation of GIC cement. Accordingly, ‘PCS coated glass particles’ are referred to as ‘PCS coated glass ionomer particles’ or ‘PCS coated GIC particles’ in some embodiments and the terms are interchanged. Similarly, the term ‘cement composition’ is sometimes used for particle compositions that do not include the liquid acid. Similarly, ‘glass powder’ is sometimes referred to as ‘glass ionomer powder’ in view of its use in forming the glass ionomer cement.

In some embodiments, for cement preparation, specific scoops are used for the powder and one drop of liquid has been added. A powder/liquid (P/L) ratio of 3.6/1 is obtained.

In some embodiments, molded specimens are prepared from cement paste. According to this aspect and in one embodiment, after mixing the particle powder and the acidic liquid, while the mixture is in a paste condition, the specimens are prepared by pouring the paste in the related molds.

According to this aspect and in one embodiment, the mixed powder and liquid forms a paste. After a period of time, the paste sets and becomes hard.

Particles of this invention can be of any diameter (or other dimension) range(s) from nanometers (nm) to millimeters (mm). In some embodiments, particles of this invention are microparticles (particles with micron-sized diameter). In some embodiments, collections of glass particles used in this invention (before application of PCS-coating) comprise microparticles, nanoparticles or any combination thereof. In some embodiments microparticles are particles with a diameter ranging between 1 micron and 1000 micron. Powders or collection of particles of this invention may be monodispersed (i.e., comprise particles of the same or of similar size, i.e., the particle collection is of narrow size-distribution) or it may comprise particles with large size distribution. Embodiments that are described herein for microparticles are also applicable to particles of other dimensions.

The thickness of the PCS coating on particles of this invention ranges from 5 nm to 50 nm in one embodiment. In other embodiments, PCS coating thickness ranges from 1 nm to 100 nm, 5 nm to 100 nm, 1 nm to 1 micron, 1 nm to 100 microns. Any other coating thickness that is suitable for GIC applications is included in embodiments of this invention.

In some embodiments, coating of the particles is complete. In some embodiments the particles are fully-coated by the PCS. In other embodiments, the particles are partially-coated by the PCS. In some embodiments, the particles are more than 50% coated. In other embodiments, the particles are less than 50% coated by PCS. Collections of particles used in this invention may include fully coated particles, partially coated particles or any combination thereof.

In some embodiments, procedures used for making the innovative cements of this invention include mixing the particle powder with acidic liquid and allowing the formed mixture to set and harden.

Powder/PCS mixing times, powder/acidic-liquid mixing times, liquid compositions, powder to liquid ratio, powder to resin ration, resin compositions, acid liquid composition, setting times and mixing/setting temperatures are not restricted to a certain value. Such parameters may vary and can be chosen or set to any value as known in the art of glass/resin and of glass-ionomer cements. For example, and in one embodiment, mixing or working time of the glass particle powder and the liquid comprising the acid is 1 minute or 2 minutes or 3 minutes or it ranges between 10 sec and 10 min or between 1 min and 5 min or between 1 min and 7 min in some embodiments. Curing/setting time is 1 minute or 2 minutes or 3 minutes in some embodiments or it ranges between 10 sec and 10 min or between 1 min and 5 min or between 1 min and 7 min in some embodiments.

In some embodiments, the powder includes dry polyacid as well.

In one embodiment, the PCS-coated glass ionomer particles are mixed with a liquid comprising polyacrylic acid at a 3.6/1 powder to liquid ratio by weight (g/g).

In one embodiment, two components are described for the cement formation: Component 1: the GIC dry powder (coated/uncoated particles) Component 2: the liquid comprising the acid.

In embodiments described herein these two components are described.

In some embodiments, the liquid comprises the acid. In one embodiment, the liquid comprises polyacid dissolved in water. The molecular weight of the polyacid used may vary and can be chosen to be suitable for an appropriate cement preparation.

In one embodiment, ‘component l’, in addition to the glass particles comprises dry acid (polyacid). According to this aspect and in one embodiment, the glass particles/dry acid are mixed with a liquid that comprise acid in one embodiment or with a liquid that does not comprise acid in another embodiment. In some embodiments, the glass-ionomer cement is referred to as glass-ionomer material.

In one embodiment, this invention provides a powder, said powder comprising glass particles coated by poly-catechol-styrene (PCS). In some embodiments, the glass is or comprises silica. In some embodiment, the glass comprises aluminosilicate. In one embodiment, the glass comprises fluoroaluminosilicate (FAS). In some embodiments, the powder comprises glass particles that are fully-coated by PCS, particles that are partially coated by PCS, particles that are not coated by PCS or any combination thereof. Powder is also referred to as a collection of particles in embodiments of this invention.

In some embodiments, this invention comprises a kit for the preparation of glass ionomer cement of the invention. In some embodiments, the kit comprises two vessels (containers). A first container comprises PCS-coated particles and a second container comprises a liquid, the liquid comprising polyacid.

In one embodiment, the first vessel that comprises a powder, comprises PCS-coated particles. In one embodiment, the powder comprises glass particles that are fully-coated by PCS, particles that are partially coated by PCS, particles that are not coated by PCS or any combination thereof in which at least some particles are partially or fully coated by PCS.

In some embodiments, the kit further comprises one or more of: liquid dispensing tools or elements, powder measuring tools or elements, a surface on which mixing can take place, mixing bowl/container, support for the containers, mixing tools or elements. In some embodiments, the liquid dispensing tool and/or the powder measuring tool is/are associated with the relevant containers, e.g., the liquid container is a squeeze bottle from which drops of liquid are dispensed in a controlled manner Another example is a container for the powder, in which the cap/lid of the container serves as the powder measuring tool.

In some embodiments, kits of this invention comprise a container comprising a resin. According to this aspect and in one embodiment, measuring/dispensing tools/elements are included in the kit for measuring/dispensing the resin.

In one embodiment, this invention provides a glass particle coated by PCS. In one embodiment, this invention provides a glass particle coated by poly-catechol-styrene (PCS), wherein the thickness of the PCS coating ranges between 1 nm and 100 nm or between 5 nm and 50 nm in some embodiments.

For many uses as described herein above, the cement preparation and its use are conducted at room temperature. However, it is to be noted that cement formation/preparation and cement use can be done at other temperatures, higher or lower than room temperature. Room temperature is usually around 18-25° C. but can be defined as any temperature between 20-30° C., 10-30° C., 0-40° C., (−10)-40° C., etc.

In one embodiment, glass particles of this invention are ball-shaped or circular shaped and their size is defined by their diameter. However, particles in powders of this invention can be of any shape including rod-like particles, other elongated particles, non-symmetric ball-shaped particles, polyhedral, rectangular, cube-shaped, oval-shaped, or particles of any other form, including symmetric, non-symmetric or partially symmetric particles, particles with smooth surface, particles with rough surface or any combination thereof.

In one embodiment, this invention provides a process for making poly-catechol-styrene-coated fluoro-aluminate silicate (FAS) glass ionomer particles.

In one embodiment, the process comprising: dispersing glass particles in a buffer; optionally ultrasonicating the dispersion; adding PCS hydrochloride to the dispersion; stirring the dispersion; collecting the particles from the dispersion; optionally washing the particles; optionally drying the particles.

In one embodiment, the buffer is mM tris(hydroxymethyl)aminomethane (TRIS) buffer. In one embodiment, the pH of the buffer is (pH=8.5). In one embodiment, the dispersion is formed, or the formation of the dispersion is facilitated by the use of vortexing. In one embodiment, ultrasonication is conducted at room temperature. In one embodiment, ultrasonication is used for 10 min or from 30 sec to 20 min. In one embodiment, stirring is applied for 4-16 h or for 1 h to 24 h. In one embodiment, stirring is conducted at room temperature. In one embodiment, stirring is conducted in the dark. In one embodiment, particles are collected by centrifugation. In one embodiment, centrifugation is performed at 300×g for 5 min. In one embodiment, the collected particles are washed with distilled water. In one embodiment, the collected particles are washed triple times with milli-Q® water (>15 M Ω). In one embodiment, the collected particles are dried at a vacuum oven. In one embodiment, in the process for making poly-catechol-styrene-coated glass ionomer particles, the weight ratio of glass particles to PCS that are mixed to form the coated particles is 10:1, 20:1, 1:1. In one embodiment, the glass particles to PCS (glass:PCS) weight ratio in preparations for producing PCS coated particles, ranges between 20:1 and 1:20, between 1:1 and 100:1, between 50:1 and 10:1. Between 100:1 and 1:1 between 1000:1 and 1:1 between 1000:1 and 1:10 between 10,000:1 and 1:1, between 20:1 and 1:1 between 50:1 and 1:1.

In one embodiment, this invention provides a method of using the cement compositions of this invention in dentistry. According to this aspect and in one embodiment, the method comprising mixing the glass particles coated by poly-catechol-styrene (PCS) with glass particles not coated by poly-catechol-styrene (PCS) to form a powder mixture. The powder mixture is then mixed with a liquid comprising a polyacid, the so formed mixture is then disposed at an in vivo site.

According to this aspect, the mixture formed from the glass particles (coated, uncoated or both) and the liquid comprising the acid is in the form of a paste. In one embodiment, this mixture is initially in the form of a paste and when it hardens it is in the form of a solid or in the form of a hardened paste. In one embodiment, the mixture is in the form of a gel.

In one embodiment when referring to particles or to powder of particles that are/is mixed with a liquid to form a cement, the liquid is a liquid comprising an ionomer, a liquid comprising an acid, a liquid comprising a polyacid, a liquid comprising a polyacid that is an ionomer. In other embodiments, the liquid does not comprise an acid, a polyacid or an ionomer.

In some embodiments, use of particles or powders of this invention for dentistry does not require mixing of the particles with any liquid.

In some embodiments, particle compositions of this invention are mixed with a liquid to form cements. In some embodiments, particle compositions of this invention are incorporated in a resin.

In some embodiments, the particles, compositions and methods described herein above for dentistry are applied to other applications such as orthopedic applications (e.g., as a bone cement for expedited fracture healing and bone regeneration/repair for skeletal defects). In some embodiments, the glass ionomer cement of this invention acts as a bone restorative material/cement. In one embodiment, powders, compositions and particles of this invention provides a new class of materials used as bone restorative material.

### EXAMPLES

### Example 1: Materials and Methods

In all these experiments, the invented restorative material is compared to Fuji IX GP®® (commercially available GIC), which is the gold standard glass-ionomer restorative cement used routinely in everyday dental practices. The commercially available GIC particles are used for the process of coating by PCS. Following the PCS coating process, the coated particles (or coated and uncoated particles) are mixed with the Fuji IX GP®® liquid to form the cement.

### Example 2: Fabrication of Poly-Catechol-Styrene (PCS) Coated Glass-Ionomer Particles

To make poly-catechol-styrene-coated fluoro-aluminate silicate (FAS) glass ionomer particles, 100 mg of glass ionomer particles are dispersed in 50 ml of mM tris(hydroxymethyl)aminomethane (TRIS) buffer (pH=8.5) using vertexing for 2 min followed by ultrasonication at room temperature for 10 min. After adding the appropriate amount of catechol-styrene-hydrochloride (10-100 mg), the mixture is stirred for 4-16 h at room temperature in dark. Time of mixing and initial catechol-styrene concentration together control the deposition amount and coating thickness. Particles are collected by centrifugation at 300×g for 5 min and washed triple times with milli-Q®® water (>15 M. Ω.) and dried at vacuum oven. RAFM is used for measuring the thickness of the PCS layer on the particles. PCS content per mg of GIC powder is evaluated using BCA assay.

### Example 3: Mineralization Capacity of PCS Containing Glass Ionomer Cements

To study whether the developed glass ionomer cement has mineralization capacity, disc shaped PCS containing glass ionomer samples with 10 mm diameter and 1 mm thickness are prepared. The particles are coated by PCS as described in Example 2 herein above. The coated particles are then mixed with the polyacid (liquid). Powder/liquid ratio is 3.6/1. The samples are immersed in simulated body fluid (SBF) or artificial saliva solutions (0.2 mM MgCl₂, 1 mM CaCl₂H₂O, 20 mM HEPES buffer, 4 mM KH₂PO₄, 16 mM KCl, 4.5 mM NH₄Cl, 300 ppm NaF, pH 7.0, adjusted with 1 M NaOH) at 37° C. for 1 and 7 days. The mineral deposition on the surface of the samples can be analyzed using SEM, EDX, and FTIR.

### Example 4: Restoration Properties Analysis

Tooth slice preparation Human third molars with and without caries are selected. Slices 0.1-0.2 cm thick are cut longitudinally using a water-cooled low speed diamond saw. To simulate early caries lesions tooth slices are acid etched with 30% phosphoric acid for 30 s and rinsed with deionized water.

The PCS coated glass ionomer particles are mixed with polyacrylic acid at a 3.6/1 powder to liquid ratio. The polyacrylic acid used has 55 kDa molecular weight. The powder and the liquid are mixed for 30-45 seconds and the material is allowed to set.

The PCS coated particles are applied on the surface of teeth with and without carious lesions. The tooth slices/PCS containing GIC are then immersed in 30 ml of artificial saliva (AS) solution (0.2 mM MgCl₂, 1 mM CaCl₂H₂O, 20 mM HEPES buffer, 4 mM KH₂PO₄, 16 mM KCl, 4.5 mM NH₄Cl, 300 ppm NaF, pH 7.0, adjusted with 1 M NaOH) at 37° C. for 1 and 7 days. After the allotted time the tooth slice is removed from the solution, rinsed with running deionized water and air dried. Rinsing does not remove the GIC from the tooth and it remains bonded to the tooth structure. SEM analysis can be utilized to study the restoration capacity of the experimental glass ionomer (PCS containing GIC) in comparison to Fuji IX® as a control group.

### Example 5: Mechanical Properties Measurement

In order to prepare PCS-containing glass powders, an appropriate amount (glass pow-der/PCS ratio of 20:1 by wt.) of glass ionomer powder, and PCS is accurately weighed, and glass ionomer particles are surface coated by stirring GIC powder in PCS solution overnight. The glass powder is Fuji IX® GIC (GC).

The PCS-coated particles (powder) are mixed with acidic liquid. A powder/liquid (P/L) ratio of 3.6/1 is used to make the set cement as recommended by the manufacturer for the uncoated GIC. The GIC specimens are mixed and fabricated at room temperature according to the manufacturer's instructions.

The powder is measured using a measuring scoop and one drop of liquid (p/l ration: 3.6/1) is added to the powder on a mixing pad. The powder and liquid are mixed for 30 seconds and the formed paste is added to the molds until the cement is set and hard.

Cylindrical specimens are prepared using cylindrical shaped molds 4 nm in diameter and 6 mm in height for compressive strength test. For the flexural strength test, cylindrical molds with 2 mm thickness, 10 mm length, and 2 mm height are used in order to prepare disc shaped samples with 10 mm diameter and 1 mm thickness. The molds are filled with the material and covered with a tape and glass slides, flattened and gently pressed by hand in order to remove air bubbles from uncured cement paste. The specimens are removed from the molds after 30 min and conditioned in distilled water at 37° C. for 1 day (23.5 h) and 7 days. Six specimens are made for each test.

Mechanical tests are performed on a screw-driven mechanical testing machine (Instron) with a crosshead speed of 0.5 mm min-1. The compressive strength is calculated. For the flexural strength test, each specimen is placed on an 8 mm diameter annular knife-edged support ring (Instron), and the load to fracture at the rate of 0.5 mm min-1, using a 3 mm diameter ball ended indenter in a universal load testing machine, is recorded. Each specimen is tested at least six times.

### Example 6: Shear Bond Strength Measurement

In order to measure the bond strength, human extracted or impacted permanent third molars are stored and surface treated according to the previous procedures. The treated teeth are then mounted in resin holders and both buccal and lingual surfaces of each tooth are trimmed with a low-speed trimmer. Subsequently, median grit silicon carbide papers (Grade P600, 1500) are used to obtain smooth dentin surfaces. Both the PCS-GIC group and the control group (Fuji IX® cement samples are mixed according to the manufacturer's instructions (instructions for the uncoated particles are applied for the coated particles as well). The samples are placed in a material holder (3.0 mm diameter×3.0 mm height). The samples are fitted by placing them in contact with the prepared dentin surfaces. The specimen assembly is then stored in 100% relative humidity at 37° C. for 1 h and then in distilled water for periods of 1 day, 7 days and 30 days.

After time intervals of 1 and 7 days of storage in distilled water, a shear load is applied to the glass ionomer/dentin interface using a standard mechanical testing machine with a knife-edged rod. All the mechanical testing machines are calibrated prior to starting the measurements. The shear force required to separate the cylinder from the dentine is recorded in Newtons and divided by the contact surface area, to determine the shear bond strength value in MPa. The debonded surfaces of the specimens are air dried and the mode of failure can be determined using an SEM. The failure mode is classified according to one of following types: adhesive, cohesive in the cement, cohesive in dentin or mixed mode of failure.

### Example 7: Microhardness Measurements

The Vickers hardness of the PCS-containing glass-ionomer samples is determined according to previously-reported methods, using a microhardness tester (Model MVK-E, M 400, Leco, St. Joseph, Mich., USA). A diamond indenter with 100 g load and a dwell time of 10 s are employed. Each of the five samples is indented two times, and the Vickers hardness number for each sample is calculated. The Vickers hardness values of the Fuji IX GP® samples are evaluated and used as the control.

### Example 8: Characterization of Working Time and Initial Setting Time

The working and setting times are determined according to a method previously utilized (see below). After mixing the glass ionomer powder and liquid (in a 3.6/1 powder to liquid ratio), a small amount of the cement is mixed for 20 s, then placed between the plates of a rheometer and allowed to set. The working and setting times are determined by calculating the time taken to reach 95% and 5% of the initial amplitude of the oscillation.

The rheometer has a spring that oscillates. When the unset cement is placed on the plate, the spring can move to 100% limit. When the cement starts setting (hardening) the spring starts to move less. When the amount of moving of the spring became 95% of original, it is the working time and when it is reduced to only 5% of original swing the cement is set.

### Example 9: Nano-Indentation Analysis-AFM

The efficiency of PCS containing GIC samples on the restoration of the carious dentin is analyzed. Nano-hardness of carious dentin before after application of PCS containing GIC are evaluated. Briefly, the PCS coated glass ionomer particles are mixed with polyacrylic acid and are applied on the surface of teeth with and without carious lesions. The tooth slices/PCS containing GIC are then immersed in 30 ml of artificial saliva (AS) solution (0.2 mM MgCl₂, 1 mM CaCl₂H₂O, 20 mM HEPES buffer, 4 mM KH₂PO₄, 16 mM KCl, 4.5 mM NH₄Cl, 300 ppm NaF, pH 7.0, adjusted with 1 M NaOH) at 37° C. for 1 and 7 days. After the allotted time the tooth slice is removed from the solution, rinsed with running deionized water for 50 s, air dried, and surface of the samples are polished using SiC abrasive papers from 800 up to 4000 grit. An atomic force microscope (AFM Nanoscope V, Digital Instruments, Veeco Metrology group, Santa Barbara, Calif.) is used to analyze the surface nano-hardness. Multiple indentations are done at different locations for carious dentin before and after PCS-GIC applications (at 3-time intervals: 1, 7, and 30 days).

### Example 10: Microtensile Bond Strength (μTBS) Test

In order to measure the microtensile bond strength, human permanent third molars buccal and lingual surfaces of each tooth are trimmed with a low-speed trimmer. Subsequently, median grit silicon carbide papers (Grade P600, 1500) are used to obtain smooth dentin surfaces. Both the PCS-GIC group and the control group (Fuji IX) cement samples are mixed according to the manufacturer's instructions and put into a material holder (3.0 mm diameter×3.0 mm height). The specimen assembly is then stored in 100% relative humidity at 37° C. for 1 h and then in distilled water for periods of 23 h, 7 days and 30 days. Samples are tested using a microtensile bond strength-testing machine (Instron 4411, Instron Corporation, Canton, Mass., USA) at a crosshead speed of 0.5 mm/min. Bond strength data are calculated in MPa.

### Example 11: Biocompatibility and Dentinogenesis Properties of PCS Containing Glass Ionomer Cements

PCS-GIC disk-shaped samples with 8 mm diameter with different ratios of PCS are fabricated, as discussed earlier. The specimens are sterilized with UV-Light for 1 hour. The sterilized disks are then used to study cellular behavior. Passage 5 Human dental pulp stem cells (DPSC) are cultured on the sterilized either PCS-GIC or control GIC (Fuji IX®) disks in regular cell culture media (a MEM, 15% FBS, 100 U/ml pen/strep, 2 mM Glutamax, 0.1 mM L-ascorbic acid) for two weeks.

Viability of cells is measured after 1 week and two weeks of incubation using LIVE/DEAD® Viability/Cytotoxicity Kit, for mammalian cells (Invitrogen). After two weeks, the odontogenic differentiation of the DPSCs cultured on PCS-GI with different ratios are studied using qPCR. Fold change in expression of dentin sialophosphoprotein (DSPP), dentin matrix protein 1 (DMP-1) and matrix extracellular phosphoglycoprotein (MEPE) are measured as markers of odontogenic differentiation. All of the experiments are repeated for 3 times. The data confirm the biocompatibility of the inventive cement. Additionally, in the presence of PCS containing GIC, PDSC are differentiated toward odontogenic (dentin-like tissues). Different ratios of PCS as noted herein above refer to the amount of coated GIC particles from the total of coated and uncoated particles.

1. A glass ionomer cement composition comprising fluoroaluminosilicate glass particles coated with a catechol-containing thin-film comprising monomeric, oligomeric, and/or polymeric catechol, semi-quinone, and/or quinone.

2. The glass ionomer cement composition as recited in claim 1, wherein the catechol-containing thin-film comprises poly catechol-styrene.

3. The glass ionomer cement composition of claim 1, wherein the thickness of the catechol-containing thin-film ranges between 5 and 100 nanometers in thickness.

4. The glass ionomer cement composition of claim 1, further comprising fluoroaluminosilicate glass particles not coated by a catechol-containing thin-film.

5. (canceled)

6. The glass ionomer cement composition of claim 3, further comprising polyacid.

7. (canceled)

8. The glass ionomer cement composition of claim 3, further comprising a resin.

9. (canceled)

10. The glass ionomer cement composition of claim 8, wherein the catechol-containing film-coated or the poly-cate-chol-styrene-coated fluoroaluminosilicate glass particles are disposed within said hydroxyethyl methacrylate resin.

11. The glass ionomer cement composition of claim 10, wherein said hydroxyethyl methacrylate resin is light cured.

12. The glass ionomer cement composition of claim 1, wherein the composition exhibits a setting time of less than 5 minutes at 25° C.

13. The glass ionomer cement composition of claim 3, wherein said glass further comprises calcium, sodium, phosphate or a combination thereof.

14. The glass ionomer cement composition of claim 3, wherein said catechol-containing thin-film comprises polycatechol-styrene HCl.

15. A method of using the glass ionomer cement composition of claim 3 in dentistry, the method comprising: (a) mixing said fluoroaluminosilicate glass particles coated by a catechol-containing thin-film with said fluoroaluminosilicate glass particles not coated by a catechol-containing thin-film to form a powder mixture; and (b) disposing the formed mixture at an in vivo site.

16. The method of claim 15, further comprising: mixing the powder comprising a catechol-containing thin-film-coated glass particles with a liquid comprising polyacid to form a cement; loading a crown or restoration with said cement; placing said crown or restoration in a patient's mouth; such that said cement composition acts as a luting element.

17. The method of claim 15, further comprising applying said cement to a tooth such that said cement composition acts as a material to bond to carious lesions, a material to reduce teeth sensitivity, as a material to promote mineralization of teeth white spots or to any combination thereof.

18. A method of using the composition of claim 3 to generate a layer of hydroxyapatite at the surface of a tooth, said method comprising: disposing the catechol-containing thin-film-coated fluoroaluminosilicate glass particles of claim 1 in a resin to form a resin/particle composition; applying the resin/particle composition to said surface of said tooth.

19. The method of claim 18, further comprising curing the resin/particle composition by light irradiation.

20. (canceled)

21. (canceled)

22. A glass ionomer cement composition comprising fluoroaluminosilicate glass particles coated with a polymeric thin-film comprising monomeric, oligomeric, and/or polymeric catechol, semi-quinone, and/or quinone.

23. The glass ionomer cement composition as recited in claim 22, wherein the polymeric thin film comprises poly catechol-styrene.

24. The glass ionomer cement composition of claim 22, wherein the thickness of the polymeric thin-film ranges between 5 and 100 nanometers in thickness.

25. The composition of claim 22, further comprising fluoroaluminosilicate glass particles not coated by a polymeric thin-film.

26. (canceled)
